(19) United States
(12) Patent Application Publication
(10) Pub. No.: US 2011/0105387 A1
(43) Pub. Date: May 5, 2011

(54) Method of treatment with rapamycin

(76) Inventors: Nian Wu, North Brunswick, NJ (US); Brian Charles Keller, Antioch, CA (US)

(21) Appl. No.: 12/924,038

(22) Filed: Sep. 18, 2010

Related U.S. Application Data
(60) Provisional application No. 61/276,953, filed on Sep. 19, 2009.

(51) Int. Cl. A61K 38/13; A61K 31/436; A61P 31/10; A61P 35/00; A61P 37/06

(52) U.S. Cl. 514/3.6; 514/291; 514/20.5; 514/19.9

(57) ABSTRACT

The invention comprises a method of treatment using an oral capsule dose of rapamycin formulated with PEG-lipids. Two types of PEG-lipids are used in the formulation. A solubilizing agent dissolves the rapamycin, and solidifying agent is used to convert the solution of rapamycin/solubilizing agent to a more solid form.

### CLAIM OF PRIORITY

This application claims priority to U.S. provisional patent application 61/276,953 entitled “Pharmaceutical Compositions of Rapamycin” filed Sep. 19, 2009.

### FIELD OF THE INVENTION

This invention relates to methods for improving the solubility and the pharmacokinetic profile of rapamycin. More particularly, the present invention relates to treatment methods employing mono or diacyl lipid-polymer conjugates for formulating rapamycin compositions having increased solubility and enhanced delivery.

### BACKGROUND OF THE INVENTION

Rapamycin is a macrolide antiobiotic produced by Streptomyces hygroscopicus which was discovered first for its properties as an antifungal agent. It adversely affects the growth of fungi such as Candida albicans and Microsporum gypseum. Rapamycin is an antibiotic that blocks a protein involved in cell division and inhibits the growth and function of certain T cells of the immune system involved in the body's rejection of foreign tissues and organs. It is a type of immunosuppressant and also a type of serine/threonine kinase inhibitor. The drug is clinically used to prevent the rejection of organ and bone marrow transplants by the body.

Delivery of hydrophobic drug compounds to the site of action is an ongoing challenge in clinical research. It has been reported that up to 40% of new chemical entities in clinical and development are water insoluble or poorly soluble [C. A. Lipinski, J Pharmacol Toxicol Method 44 (2000) 235-2490 and N. Gursoy and S. Benita, Biomed. Pharmacother. 58 (2004) 173-182]. Rapamycin is insoluble in water and is only slightly soluble in solubilizers commonly used in preparing parenteral formulations such as propylene glycol, glycerin and PEG 400. Cyclodextrins, drug-lipid complexes, liposomes, and other solubilizing agents such as Cremophor® and various PEG-lipid conjugates have been tested as the delivery vehicles for rapamycin. However, no significantly improvement in pharmacokinetic profiles and bioavailability are achieved in these vehicles. It is therefore an object of this invention to present new compositions and methods for formulating rapamycin in various dosage forms.

### BRIEF DESCRIPTION OF THE INVENTION

The invention comprises a method of treatment using an oral capsule dose of rapamycin formulated with PEG-lipids. Two types of PEG-lipids are used in the formulation. A solubilizing agent dissolves the rapamycin, and solidifying agent is used to convert the solution of rapamycin/solubilizing agent to a more solid form.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

In the drawings:

FIG. 1 shows pharmacokinetic profiles of rapamycin formulations after IV dosing.

FIG. 2 shows pharmacokinetic profiles of rapamycin formulations after oral dosing.

### DETAILED DESCRIPTION

Embodiments of the present invention are described herein in the context of mono or diacyl lipid-polymer conjugates for increasing the solubility and enhancing the delivery of rapamycin. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

U.S. Pat. Nos. 6,610,322, 6,958,160, and 7,150,883, which are hereby incorporated by reference, teach the formation of spontaneous liposomes by employing certain diacylglycerol-polyethleneglycol (DAG-PEG) conjugates. The patents described how to select PEG-lipid conjugates which form liposomes by simply adding the conjugate to an aqueous solution. It now has been discovered that similar polymer-lipid molecules are useful for solubilizing hydrophobic drugs without the formation of liposomes. Both the spontaneous liposome technology and solubilization without the formation of liposomes by DAG-PEGs may be employed in new formulations of rapamycin.

Rapamycin is a macrocyclic lactone produced by Streptomyces hygroscopicus [Singh, K., Sun, S. & Vezina, C., J. Antibiot. (Tokyo), 32 (1979) 630-645]. It is poorly soluble in water and therefore can only be given orally. It is currently available in both liquid and tablet formulations. Although the tablet formulation resulted in a lower maximum concentration (Cₘₐₓ), the area under the concentration-time curves (AUCs) of the two formulations are similar [Kelly, P. A., Napoli, K. & Kahan, B. D., Biopharm. Drug Dispos., 20 (1999) 249-253]. The peak concentrations of rapamycin can be quickly attained within 2 hours after oral dosing, however its bioavailability is relatively low (˜15%) [Napoli, K. L. & Taylor, P. J., Ther. Drug Monit. 23 (2001) 559-586] and also exhibits wide interpatient variability. The effects of intestinal cytochrome P450 3A enzymes (CYP3A) and P-glycoprotein activity on rapamycin absorption are believed to be largely attributed to this variability [Mahalati, K. & Kahan, B. D., Clin. Pharmacokinet., 40 (2001) 573-585].

While rapamycin offers promising pharmacological activities, its poorly water solubility and low bioavailability are the two major pharmacokinetic limitations [Montaguti, P., Melloni, E., Cavalletti, E., Arzneimittelforschung, 44 (1994) 566-570; Sehgal, S. N., Baker, H., Vezina, C., J Antibiot (Tokyo), 28 (1975) 727-732]. Historically many attempts to develop intravenous formulations have been unsuccessful [Simamora, P., Alvarez, J. M., Yalkowsky, S. H., Int J. Pharm., 213 (2001) 25-29], therefore only oral solutions and tablet forms are clinically available [Mahalati, K., Kahan, B. D., Clin Pharmacokinet., 40 (2001) 573-585].

The low oral bioavailability (<15%) of rapamycin does limit its therapeutic applications [Napoli, K. L., Wang, M. E., Stepkowski, S. M., Kahan, B. D., Clin Biochem., 30 (1997) 135-142; Trepanier, D. J., Gallant, H., Legatt, D. F., Yatscoff, R. W., Clin Biochem., 31 (1998) 345-351; Yatscoff, R. W., Wang, P., Chan, K., Hicks, D., Zimmerman, J., Ther Drug Monit., 17 (1995) 666-671] except for low-dosage treatments such as immunosuppression in renal and liver transplant recipients [Maramattom, B. V., Wijdicks, E. F., Neurology, 63 (2004) 1958-1959]. Sensitivity to gastric acid, partial intestinal absorption and loss from first-pass hepatic metabolism (<3% excreted in urine) [Kahan, B. D., Ther Drug Monit., 24 (2002) 47-52] all contribute to rapamycin's low bioavailability.

Rapamycin is also considered as an anticancer agent. However, its strong partition to erythrocytes may hinder accessibility into solid tumor sites [Trepanier, D. J., Gallant, H., Legatt, D. F., Yatscoff, R. W., Clin Biochem., 31 (1998) 345-351; Yatscoff, R. W., Wang, P., Chan, K., Hicks, D., Zimmerman, J., Ther Drug Monit., 17 (1995) 666-671; Tu, Y., Stepkowski, S. M., Chou, T. C., Kahan, B. D., Transplantation, 59 (1995) 177-183; Mahalati, K., Kahan, B. D., Clin Pharmacokinet, 40 (2001):573-585; Yatscoff, R., LeGatt, D., Keenan, R., Chackowsky, P., Transplantation, 56 (1993) 1202-1206].

Different approaches have been taken to overcome these limitations and improve the formulation and delivery of rapamycin including a water-soluble prodrug of rapamycin, CCI-779 [Drugs RD., 5 (2004) 363-367]. The water solubility of CCI-779 is only slightly improved (□ 120 μg/ml) and ethanol is used as a co-solvent for IV formulations [Raymond, Em, Alexandre. J., Faivre, S., Vera, K., Materman, E., Boni, J., Leister, C., Korth-Bradley, J., Hanauske, A., Armand, J. P., J Clin Oncol., 22 (2004) 2336-2347; Montaguti, P., Melloni, E., Cavalletti, E., Arzneimittelforschung, 44 (1994) 566-570]. In addition, CCI-779 prodrug is rapidly hydrolyzed by plasma esterases back into rapamycin which redistribute and partition into blood erythrocytes [Raymond., E., Alexandre, J., Faivre, S., Vera, K., Materman, E., Boni, J., Leister, C., Korth-Bradley, J., Hanauske, A., Armand, J. P., J Clin Oncol., 22 (2004) 2336-2347; Yatscoff., R. W., Wang, P., Chan, K., Hicks, D., Zimmerman, J., Ther Drug Monit., 17 (1995) 666-671] and may reduce accumulation in solid tumor sites. Furthermore, some of the side effects observed from clinical trials are exacerbated compared to the observed side effects after rapamycin administration. Such side effects include headache and abdominal pain, [Ettenger, R. B., Grimm, E. M., Am J Kidney Dis., 38 (2001) S22-S28; Tejani, A., Alexander, S., Ettenger, R., Lerner, G., Zimmerman, J., Kohaut, E., Briscoe, D. M., Pediatr Transplant., 8 (2004) 151-160], skin disorders, nocturnal calf cramps, and muscle aching [Finsterer, J., Kanzler, M., Weinberger, A., Transplantation, 76 (2003) 1773-1774]. Therefore, it is necessary to explore other alternatives in formulation development to improve the pharmacokinetic and biodistribution profile, minimize the preferential partition of rapamycin into erythrocytes, and eliminate some of the toxic side-effects.

It has been reported that coadministration of cyclosporine on renal transplant patients may improve the bioavailability of rapamycin if the drugs are administered concomitantly. Both the Cₘₐₓ and the AUC of rapamycin are increased due to the inhibition of cyclosporine on CYP3A4 and P-glycoprotein [Kaplan, B., Meier-Kriesche, H. U., Napoli, K. L. & Kahan, B. D., Clin. Pharmacol. Ther., 63 (1998) 48-53]. It has also been observed that a high-fat meal can significantly affect the absorption of rapamycin with a 35% increase in AUC at a slower rate of the absorption [Zimmerman, J. J., Ferron, G. M., Lim, H. K. and Parker, V., J. Clin. Pharmacol., 39 (1999) 1155-1161].

One aspect the present invention is to comprise both rapamycin and cyclosporine in an aqueous or solid based formulation in which a single diacyl lipid-polymer conjugate is used as the solubilizing agent and/or bioavailability enhancer. The cyclosporine content is preferably equal to or less than the concentration of rapamycin in the case of such coadministration, with the ratio of cyclosporine to rapamycin in a range of 0.1 to 1.

A preferred embodiment of the present invention comprises an aqueous-based, injectable rapamycin solution including 1,2-dimyristoyl-rac-3-monomethoxydodecamethylene glycol (mPEG-12)-glycerol (GDM-12) or 1,2-dioleoyl-rac-3-monomethoxydodecamethylene glycol (mPEG-12)-glycerol (GDO-12) and a buffer. Preferably, the solution includes rapamycin in concentrations ranging from 0.05 mg/mL to 50 mg/mL and the ratio of PEG-lipid to the drug ranges from 0.2 to 25. More preferably, the concentration of rapamycin ranges from 1.0 mg/mL to 10 mg/mL. Most preferably, the concentration of rapamycin ranges from 1 mg/mL to 5 mg/mL and the percent of PEG-lipid ranges from 0.5 to 10 (w/v) of the total solution.

Further preferable aqueous, injectable rapamycin solutions of the invention are those in which the diluent consists of 0.5 to 25 percent (w/v) of the PEG-lipid and 75 to 99.5 percent (v/v) of water or a buffer or saline solution. Also preferable are aqueous, injectable rapamycin solutions of this invention in which 80 to 99 percent (v/v) of the total solution is water or a buffer or saline solution.

The most preferable aqueous injectable rapamycin solutions according to the present invention comprise rapamycin in GDM-12 or GDO-12 plus aqueous buffer at concentrations of rapamycin ranging from 0.5 mg/mL to 50 mg/mL, 2.5 to 25 percent (w/v) of GDM-12 or GDO-12, and 75 to 98 percent (v/v) water, wherein the concentration of rapamycin in the combined solution ranges from 0.5% to 5%.

The aqueous injectable rapamycin solutions of this invention can be administrated by bolus injection or by infusion. Infusion is preferable for such solutions where the concentration of rapamycin in is greater than 0.1 mg/mL. In case of an infusion, the length of an infusion is preferable 30 minutes to 6 hours and should not be more than 24 hours.

Another aspect of the present invention is an aqueous oral solution of rapamycin comprising rapamycin in the range of 0.5 mg/mL to 10 mg/mL. Preferred aqueous, oral rapamycin solutions are those wherein one or more of the PEG-lipids from Table 1 is included.

Preferable aqueous oral rapamycin solutions of this aspect of the invention are those wherein the concentration of rapamycin in the solution ranges from 0.1 mg/mL to 10 mg/mL. Also preferred are those solutions wherein the PEG-lipid comprises about 0.5 to 20 weight percent of the solution, and water comprises about 80 to 99.5 percent by volume of the total solution.

Yet another aspect of the present invention is an oral capsule of rapamycin comprising rapamycin in the range of 0.5 mg/capsule to 10 mg/capsule. Preferred oral capsules of rapamycin are those wherein two of the PEG-lipids are present as selected from Table 1.

In the capsule dosage form, a short PEG-chain lipid is used as a solublizing agent and a long PEG chain lipid is used as the GI absorption enhancer and solidifying agent.

Preferable oral capsules of rapamycin of this aspect of the invention are those wherein the amount of rapamycin ranges from 0.5 mg/capsule to 10 mg/capsule. Also preferred are those wherein the PEG-lipid comprises 95 to 99 weight percent of the capsule content.

The present invention involves solubilizing rapamycin, or rapamycin plus cyclosporine, by using one or more amphipathic PEG conjugates. Diacylglecerol-polyethyleneglycols (DAG-PEGs) are preferred solubilizing agents, in which acyl chains comprise the lipophilic portion of the conjugate.

A bis polyethyleneglycol-monoacylglecerol (BisPEG-MAG) can also be an excellent solubilizing agent, in which the single acyl or chain comprise the lipophilic portion of the conjugate. Similarly, PEG mono and dicholylglycerols can also be used as solubilizing agents. As with DAG-PEG solubilizing agents, these compounds must be liquid at the temperature of solubilization, so compounds with melting points below about 25 degrees C. are preferred. Such solubilizing agents can be used to prepare IV formulations, oral liquids and oral capsules.

The preferred first step for solubilization is combining the drug compound(s) with an amphipathic PEG conjugate which is liquid at the temperature of solubilization. For formulating at room temperature (which is preferred), this means employing a conjugate having a melting temperature less than about 25 degrees Centigrade. Such solubilization is preferably done by first adding the drug to the conjugate only. If an aqueous solution is desired, the aqueous solution is later mixed with the drug/lipid mixture. Alternatively, if an aqueous suspension is desired, the drug compound(s) may be added to a mixture of the DAG-PEG in aqueous solution.

For applications where a solid or semi-solid form is more desirable (i.e., oral capsule), a solidifying agent having a higher melting temperature is added after the initial solubilization. The solidifying agent is preferably a second DAG-PEG having a melting temperature above room temperature. Preferably, the melting temperature of the solidifying agent is between about 35 and 65 degrees C.

By performing solubilization at elevated temperatures, conjugates with higher melting temperatures may be used as solubilizing agents. When forming aqueous solutions, the aqueous solution is also preferably added at an elevated temperature. When forming oral liquid capsules, a separate solidifying agent may not be needed. Likewise when forming oral solid capsules, a separate solubilizing agent may not be needed if the solibilizing agent is used to solubilize the drug at elevated temperatures. GDS-12, with a melting point of about 40 degrees C., is an example of a compound that may be used this way.

The DAG-PEG lipids shown in Table 1 are all suitable for use in various aspects of the present invention. DAG-PEGs with oxy or succinyl linkers (X=oxygen or succinyl) are preferred, though DAG-PEGs with other linkers may be used. The DAG-PEGs with melting temperatures less than about 25 degrees C. are suitable as solubilizing agents for solubilization at room temperature. Those with melting temperatures above about 25 degrees C. are preferably used as solidifying agents for oral capsules, though they may also be used as solubilizing agents if solubilization is performed at elevated temperatures. Preferably, solidifying agents have a melting temperature greater than about 35 degrees C. For new DAG-PEGs synthesized for use in the invention (e.g., those with linkers other than oxy), melting temperatures can be determined empirically.

TABLE 1
PEG-lipid (diacylglycerol- polyethyleneglycols) used for the invention

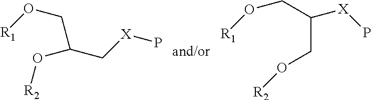

In the structure R1 and R2 are the same or different fatty acids as described in the table and P
is the PEG chain. X represents a linker which may be oxy or thiol, amino or succinyl or the
like which is not distinguished in the following name. “n” in the table below indicates the
number of subunits in the PEG polymer.

| Shorthand name | Name |
|---|---|
| GDM-12 | 1,2-dimyristoyl-rac-glycerol-3-dodecaethylene glycol, n = 12 |
| GDO-12 | 1,2-dioleoyl-rac-glycerol-3-dodecaethylene glycol, n = 12 |
| GDC-12 | 1,2-dicholoyl-rac-glycerol-3-dodecaethylene glycol, n = 12 |
| GDM-600 | 1,3-dimyristoyl-glycerol-2-dodecaethylene glycol, n = 12 |
| GDO-600 | 1,3-dioleoyl-glycerol-2-dodecaethylene glycol, n = 12 |
| GDC-600 | 1,3-dicholoyl-glycerol-2-dodecaethylene glycol, n = 12 |
| GDS-12*** | 1,2-distearoyl-rac-glycerol-3-dodecaethylene glycol, n = 12 |
| GOB-12 | 1,2-bis(dodecaethylene glycol)glycerol-3-oleate, n = 12 |
| GMB-12 | 1,2-bis(dodecaethylene glycol)glycerol-3-myristate, n = 12 |
| DSB-12 | 1,2-bis(dodecaethylene glycol)glycerol-3-stearate, n = 12 |
| GOBH | 1,2-bis(hexaethyle glycol)glycerol-3-oleate, n = 6 (x2) |
| GMBH | 1,2-bis(hexaethyle glycol)glycerol-3-myristate, n = 6 (x2) |
| GCBH | 1,2-bis(hexaethyle glycol)glycerol 3-cholate, n = 6 (x2) |
| GPBH | 1,2-bis(hexaethyle glycol) glycerol-3-palmitate, n = 6 (x2) |
| GDO-23 | 1,2-dioleoyl-rac-glycerol-3-polyethylene(1000) glycol, n = 23 |
| GDO-27 | 1,2-dioleoyl-rac-glycerol-3-polyethylene(1200) glycol, n = 27 |
| GDM-23 | 1,2-dimyristoyl-rac-glycerol-3-polyethylene(1000) glycol, n = 23 |
| GDM-27 | 1,2-dimyristoyl-rac-glycerol-3-polyethylene(1200) glycol, n = 27 |
| GDS-23 | 1,2-distearoyl-rac-glycerol-3-polyethylene(1000) glycol, n = 23 |

*** GDS-12 is a solid at 25° C. and can be used as both a solidifier and a solubilizing agent

As previously mentioned, certain DAG-PEGs (many of which are useful in practicing the present invention) spontaneously form liposomes upon mixing with an aqueous solution. Other DAG-PEGs useful in the invention do not display such property. Liposomes may be preferable solubilizing agents for IV solutions due to more predictable and homogeneous particle sizes and superior stability. However, non-liposomal formulations are also useful, especially for oral formulations.

Mixtures of DAG-PEGs may be used in the invention in the place of single species of DAG-PEGs. For example, a formulation may include GDO-12, GDM-12, or a combination of the two DAG-PEGs. When combinations of DAG-PEGs are used, the properties of the lipid mixture (e.g., melting point or average size of the PEG chain) may be calculated by known methods or determined empirically.

The manufacture of rapamycin IV solution comprises first adding the rapamycin to the PEG-lipid and mixing until homogenous, which may be accomplished at room temperatures. Next, premixed aqueous integrants are added to the lipid-rapamycin mixture and mixed until a homogenous solution is obtained. The solution is then filtered for sterility while maintaining an overlay of sterile-filtered nitrogen during the process. Appropriate volumes of the solution are filled into ampules and sealed using aseptic technique. Sterile conditions are maintained throughout the filtering, filling and sealing operations in accordance with standard manufacturing procedures for injectables. While the formulated product is stable at room temperature, it is preferably stored under refrigeration for extended shelf life.

It is an object of his invention to provide formulations of rapamycin with increased bioavailability. It is a further object of his invention to provide formulations of rapamycin with increased solubility. It is still a further object of his invention to provide formulations of rapamycin with increased stability.

In one aspect the invention is a pharmaceutical composition for administration by intravenous injection. The composition comprises an aqueous solution; a liposome-forming DAG-PEG or combination of DAG-PEGs; and rapamycin at a concentration between about 0.05 mg/ml and about 50 mg/ml. The weight ratio of the DAG-PEG to the rapamycin is preferably between about 0.2 and 25. The average MW of PEG chains in the DAG-PEG or mixture of DAG-PEGs is preferably less than about 600. The melting point of the DAG-PEG or combination of DAG-PEGs is preferably less than about 25 degrees C. The DAG-PEG may comprise 1,2-dimyristoyl-rac-3-monomethoxydodecamethylene glycol (mPEG-12)-glycerol (GDM-12) or 1,2-dioleoyl-rac-3-monomethoxydodecamethylene glycol (mPEG-12)-glycerol (GDO-12). The concentration of rapamycin is preferably between about 0.2 mg/ml to 25 mg/ml. The concentration of DAG-PEG is preferably between about 0.5 to 25 percent (w/v) of the total solution. The composition may further comprise cyclosporin, where the ratio of cyclosporin to rapamycin is between about 0.1 to 1.

In another aspect, the invention is a method of making a pharmaceutical composition suitable for administration by intravenous injection. The method comprises mixing a DAG-PEG or combination of DAG-PEGs with rapamycin; and adding an aqueous solution while mixing to create a suspension. The DAG-PEG or combination of DAG-PEGs may be selected to spontaneously form liposomes upon addition of the aqueous solution. The final concentration of rapamycin is preferably between about 0.05 mg/ml and about 50 mg/ml. The weight ratio of the total DAG-PEG to the rapamycin is preferably between about 0.2 and 25. The average MW of PEG chains in the DAG-PEG or combination of DAG-PEGs is preferably less than about 600. The melting point of the DAG-PEG or combination of DAG-PEGS is preferably less than about 25 degrees C. The DAG-PEG may comprise 1,2-dimyristoyl-rac-3-monomethoxydodecamethylene glycol (mPEG-12)-glycerol (GDM-12) or 1,2-dioleoyl-rac-3-monomethoxydodecamethylene glycol (mPEG-12)-glycerol (GDO-12). The final concentration of rapamycin is preferably between about 0.2 mg/ml to 25 mg/ml. The final concentration of DAG-PEG is preferably between about 0.5 to 25 percent (w/v) of the total solution. The method may further include mixing cyclosporin with the rapamycin and DAG-PEG or combination of DAG-PEGs, where the weight ratio of cyclosporin to rapamycin is between about 0.1 to 1. The method may further comprise sealing the aqueous suspension in a sterile container.

In another aspect the invention is a method of treating a disease in a mammal. The method comprises preparing a composition comprising an aqueous solution; a liposome-forming DAG-PEG or combination of DAG-PEGs; and rapamycin at a concentration between about 0.05 mg/ml and about 50 mg/ml. The weight ratio of the DAG-PEG to the rapamycin is between about 0.2 and 25. The composition is administered to the mammal intravenously. The average MW of PEG chains in the DAG-PEG or combination of DAG-PEGs is preferably less than about 600. The melting point of the DAG-PEG or combination of DAG-PEGs is preferably less than about 25 degrees C. The DAG-PEG may comprise 1,2-dimyristoyl-rac-3-monomethoxydodecamethylene glycol (mPEG-12)-glycerol (GDM-12) or 1,2-dioleoyl-rac-3-monomethoxydodecamethylene glycol (mPEG-12)-glycerol (GDO-12). The concentration of rapamycin is preferably between about 0.2 mg/ml to 25 mg/ml. The concentration of DAG-PEG is preferably between about 0.5 to 25 percent (w/v) of the total solution. The composition may further comprise cyclosporin, where the weight ratio of cyclosporin to rapamycin is between about 0.1 to 1. The disease may be cancer or a fungal infection. Treatment of the disease may require immunosuppression.

In another aspect the invention is a pharmaceutical composition for administration by oral solution. The composition comprises an aqueous solution; a DAG-PEG or combination of DAG-PEGs; and rapamycin at a concentration between about 0.5 mg/ml and about 10 mg/ml. The weight ratio of the DAG-PEG or combination of DAG-PEGs to the rapamycin is preferably between about 0.2 and 25. The average MW of PEG is preferably less than about 600. The total DAG-PEG concentration is preferably between about 0.5 to 25 percent (w/v) of the total solution. The composition may further comprising cyclosporin, where the weight ratio of cyclosporin to rapamycin is between about 0.1 to 1.

In another aspect the invention is a method of making a pharmaceutical composition suitable for administration by oral solution. The method comprising mixing a DAG-PEG or combination of DAG-PEGs with rapamycin; and adding an aqueous solution while mixing to create a suspension. The final concentration of rapamycin is preferably between about 0.05 mg/ml and about 50 mg/ml. The weight ratio of the total DAG-PEG to the rapamycin is preferably between about 0.2 and 25. The average MW of PEG chains in the DAG-PEG or combination of DAG-PEGs is preferably less than about 600. The melting point of the DAG-PEG or combination of DAG-PEGs is preferably less than about 25 degrees C. The final concentration of rapamycin is preferably between about 0.2 mg/ml to 25 mg/ml. The final concentration of DAG-PEG is preferably between about 0.5 to 25 percent (w/v) of the total solution. The method may further include mixing cyclosporin with the rapamycin and DAG-PEG or combination of DAG-PEGs, where the weight ratio of cyclosporin to rapamycin is between about 0.1 to 1. The method may further comprise sealing the aqueous suspension in a sterile container.

In another aspect the invention is a method of treating a disease in a mammal. The method comprises preparing a composition comprising an aqueous solution; a DAG-PEG or combination of DAG-PEGs; and rapamycin at a concentration between about 0.5 mg/ml and about 10 mg/m. The composition is administered as an oral solution. The weight ratio of the DAG-PEG or combination of DAG-PEGS to the rapamycin is preferably between about 0.2 and 25. The average MW of PEG is preferably less than about 600. The total DAG-PEG concentration is preferably between about 0.5 to 25 percent (w/v) of the total solution. The composition may further comprise cyclosporin, where the weight ratio of cyclosporin is between about 0.1 to 1. The disease may be cancer or a fungal infection. Treatment of the disease may require immunosuppression.

In another aspect the invention is a pharmaceutical composition for administration by oral capsule. The composition comprises a solubilizing agent; a solidifying agent; rapamycin; and a capsule. The solubilzing agent preferably comprises a DAG-PEG, though other compounds may be used. The MW of the PEG chain of the solubilizing agent is less than about 600. The melting point of the DAG-PEG of the solubilizing agent is less than about 25 degrees C. The solidifying agent preferably comprises a DAG-PEG, though other compounds may be used. The MW of the PEG chain of the solidifying agent is preferably greater than about 600. The melting point of the DAG-PEG of the solidifying agent is preferably greater than about 35 degrees C. The composition may further comprise cyclosporin. DAG-PEGs preferably comprise about 90 to 99.8 weight percent of the capsule content. The capsule preferably contains between about 0.5 and 25 mg of rapamycin. More preferably, the capsule contains about 0.5 to 10 mg of rapamycin. When cyclosporin is included, the cyclosporin to rapamycin weight ratio is preferably between about 0.1 and 1.0.

In another aspect the invention is a method of making a pharmaceutical composition suitable for administration by oral capsule. The method comprises mixing a solubilizing agent with rapamycin; adding a solidifying agent with further mixing; and filling the resulting mixture into a capsule. The solubilizing agent preferably comprises a DAG-PEG or combination of DAG-PEGs. The average MW of PEG chains in the DAG-PEG or combination of DAG-PEGs of the solubilizing agent is preferably less than about 600. The melting point of the DAG-PEG or combination of DAG-PEGs of the solubilizing agent is preferably less than about 25 degrees C. The solidifying agent preferably comprises a DAG-PEG or combination of DAG-PEGs. The average MW of PEG chains in the DAG-PEG or combination of DAG-PEGs of the solidifying agent is preferably greater than about 600. The melting point of the DAG-PEG or combination of DAG-PEGs of the solidifying agent is preferably greater than about 35 degrees C. The method may further include mixing cyclosporin with the rapamycin and solubilizing agent, where the weight ratio of cyclosporin to rapamycin is between about 0.1 to 1. The resulting capsule preferably contains between about 0.5 and 25 mg of rapamycin. More preferably, the capsule contains between about 0.5 and 10 mg of rapamycin.

In another aspect the invention includes a method of treating a disease in a mammal. The method comprises preparing a composition comprising a solubilizing agent; a solidifying agent; rapamycin; and a capsule. The composition is administered to the mammal orally. The solubilzing agent preferably comprises a DAG-PEG. The MW of the PEG chain of the DAG-PEG solubilizing agent is preferably less than about 600. The melting point of the DAG-PEG solubilizing agent is preferably less than about 25 degrees C. The solidifying agent preferably comprises a DAG-PEG. The MW of the PEG chain of the DAG-PEG solidifying agent is preferably greater than about 600. The melting point of the DAG-PEG solidifying agent is preferably greater than about 35 degrees C. The composition may further comprise cyclosporin. Preferably, DAG-PEGs comprise about 90 to 99.8 weight percent of the capsule content. The capsule preferably contains between about 0.5 and 25 mg of rapamycin. More preferably, the capsule contains between about 0.5 and 10 mg of rapamycin. The weight ratio of cyclosporin to rapamycin is preferably between about 0.1 to 1.0. The disease may be cancer or a fungal infection. Treatment of the disease may require immunosuppression.

The composition, method of making, and method of treatment related to the oral capsule are generally applicable to other hydrophobic drug compounds suitable for oral use.

The following examples intend to further illustrate the practice of the present invention.

### Example 1

### Preparation of Rapamycin Oral Solution

A rapamycin solution suitable for oral delivery is prepared as follows. DAG-PEG is added to a vessel equipped with a mixer propeller. The drug substance is added with constant mixing. Mixing is continued until the drug is visually dispersed. Pre-dissolved excipients in water are slowly added to the vessel with adequate mixing. Mixing continued until a homogenous solution is achieved. A sample formulation is described in Table 2.

TABLE 2

| Ingredient | mg/mL |
|---|---|
| Rapamycin | 2.0 |
| PEG Lipid | 50 |
| Organic Acid | 10 |
| Sodium Hydroxide | See below |
| Hydrochloric Acid | See below |
| Sodium Benzoate | 2.0 |
| Artificial Flavor | 5.0 |
| Purified Water | qs 1 mL |

One or more PEG lipids is selected from Table 1, where n=12 or less (i.e., the MW of the PEG chain is less than about 600). Sodium hydroxide is used to prepare a 10% w/w solution in purified water. The targeted pH is in a range of 4.0 to 7.0. The NaOH solution is used to adjust pH if necessary. The drug to lipid ratio is preferably greater than about 1 to 20, and more preferably greater than about 5 to 10. The organic acid may be lactic acid or pyruvic acid or glycolic acid, though lactic acid is most preferable. The concentration of organic acid is preferably in the range 1 and 10%, and more preferably about 2 to 5%.

### Example 2

### Preparation of Rapamycin/Cyclosporin Oral Solution

A rapamycin and cyclosporine solution suitable for oral delivery of rapamycin was prepared as follows. DAG-PEG was added to a vessel equipped with a mixer propeller. The drug substance was added with constant mixing. Mixing continued until the drug was visually dispersed in the lipid. Pre-dissolved excipients in water were slowly added to the vessel with adequate mixing. Mixing continued until fully a homogenous solution was achieved. A sample formulation is described in Table 3.

TABLE 3

| Ingredient | mg/mL |
|---|---|
| Rapamycin | 2.0 |
| Cyclosporine | 1.0 |
| PEG Lipid | 50 |
| Organic Acid | 10 |
| Sodium Hydroxide | See below |
| Hydrochloric Acid | See below |
| Sodium Benzoate | 2.0 |
| Artificial Flavor | 5.0 |
| Purified Water | qs 1 mL |

One or more PEG lipids having n=12 or less (i.e., the MW of the PEG chain is less than about 600) was selected from Table 1. Sodium hydroxide was used to prepare a 10% w/w solution in purified water. The targeted pH as in a range of 4.0 to 7.0. The NaOH solution was used to adjust pH as necessary. The drug to lipid ratio is preferably greater than about 1 to 20, and more preferably greater than about 5 to 10. The organic acid may be lactic acid or pyruvic acid or glycolic acid, though lactic acid is most preferable. The concentration of organic acid is preferably in the range 1 and 10%, and more preferably about 2 to 5%.

### Example 3

### Rapamycin IV Injectable Solution

The IV solution is prepared as in Example 1, except that the targeted pH range was between 6.0 and 7.5 and sterile conditions are maintained throughout the process. A sample formulation is described in Table 4.

TABLE 4

| Ingredient | mg/mL |
|---|---|
| Rapamycin | 10.0 |
| DAG-PEG Lipid | 100 |
| Sodium Hydroxide | See Below |
| Lactic Acid | 25 |
| Purified Water | qs 1 mL |

### Example 4

### Rapamycin/Cyclosporine IV Injectable Solution

The IV solution is prepared as in Example 2, except that the targeted pH range was between 6.0 and 7.5 and sterile conditions are maintained throughout the process. A sample formulation is described in Table 5.

TABLE 5

| Ingredient | mg/mL |
|---|---|
| Rapamycin | 4.0 |
| Cyclosporine | 2.0 |
| DAG-PEG Lipid | 50 |
| Sodium Hydroxide | as needed |
| Lactic Acid | 20 |
| Purified Water | qs 1 mL |

### Example 5

### Rapamycin Capsules

A sample formulation is described in Table 6. Rapamycin is charged to a suitable vessel equipped with a mixer propeller. Lactic acid is added with gentle mixing to levigate the drug powder. 100% of the final batch volume of first PEG-lipid (the solubilizing agent, liquid, n≦12) is added with constant mixing. Mixing is continued until the suspension is fully dispersed. The second PEG-lipid (the solidifying agent, solid, n>12) is slowly added to the vessel with constant mixing. Mixing is continued with slow agitation (above the melting point of the solidifying agent, typically about 50 to 55° C.) until the solid lipid is visually dispersed in the solution. The mixture is kept warm and transferred to the filling steps.

The appropriate filling equipment (e.g. Bosch's GKF 1400L) was set up with the required fill volume. The batch is filled into the capsules. The batch is continually agitated. No. 1 blue opaque hard gelatin capsule shells at a target fill weight of ˜215 mg are used, employing a suitable capsule machine (e.g., Bosch GKF 2000S capsule filler or Capsugel CFS 1200 or Planeta Capsule Filler). The capsules are transferred into a suitable closed cool chamber container (0 to −20° C.) over night to let the capsule content be solidified. The solidified capsules are polished using a suitable polisher (e.g., Key Turbo Kleen CP-300 Capsule Polisher). The finished capsules are transferred into a suitable closed container.

TABLE 6

| Ingredient | mg/cap |
|---|---|
| Rapamycin | 5.0 |
| Lactic acid | 10 |
| PEG Lipid (liquid) | 100.0 |
| PEG Lipid (solid) | 100.0 |

A liquid PEG lipid (n=12 or less) and a solid PEG-lipid are selected from Table 1 or any combination thereof.

### Example 6

### Pharmacokinetic Profile and Bioavailability of Rapamycin Formulations

To assess the effect of the new formulations on the basis of pharmacokinetic parameters, mixed gender Sprague-Dawley rats (200-240 g, n=3 for each treatment group) were dosed intravenously and orally (10 mg/kg) with rapamycin (a) dissolved in 25 mM of sodium phosphate buffer (pH 7) containing 10% and 5% methanol as the control formulation); (b) in PEG-lipid formulation (5% of GDO-12 in 25 mM of sodium phosphate buffer, pH=7); or (c) in PEG-lipid formulation co-incorporated with cyclosporine (3 mg/kg). The rats were given 1 mL of each formulation as IV bolus within 5 min or 1 mL of oral gavages. After dosing, blood samples were collected from the cannula at 0, 5 or 15, and 30 min, then 1, 2, 4, 6, 12, 24, 36 and 48 h after IV and oral administration, and the cannula flushed with 0.9% saline. The blood samples (0.2 ml) were collected in heparanized tubes and following centrifugation, the plasma were collected and stored at −79° C. until LC-MS analyzed.

Rapamycin levels were determined following liquid-liquid phase extraction using an HPLC/tandem mass spectrometry (LC-MS/MS). Plasma samples and calibration standards were extracted using the liquid-protein precipitation technique (acetonitrile/MeOH=4/1). Reverse-phase chromatography (a C₁₈ column of 50×4.6 mm with 5 μm particle size) was employed using a gradient elution (i.e., 50% phase A to 80% phase B in 8 minutes) with 0.1% formic acid/water and 0.1% formic acid/ACN as mobile phase A and B, respectively. The analyses were performed in electro-spray positive mode using multiple reaction monitoring conditions (ion pair of m/z 931.6 (M+NH₄)⁺/864.6) on a Sciex API-4000 (Applied Biosystems, Foster City, Calif.) instrument.

Pharmacokinetic analysis was performed using data from individual rats for which the mean and standard error of the mean (SEM) were calculated for each group. The elimination rate constant (Kₑₗ) was estimated by linear regression of the blood or plasma concentrations in the log-linear terminal phase. The pharmacokinetic parameters were estimated from a two-compartmental model which was fitted to the plasma concentration versus time data using WinNonlin® software (Version 5.5).

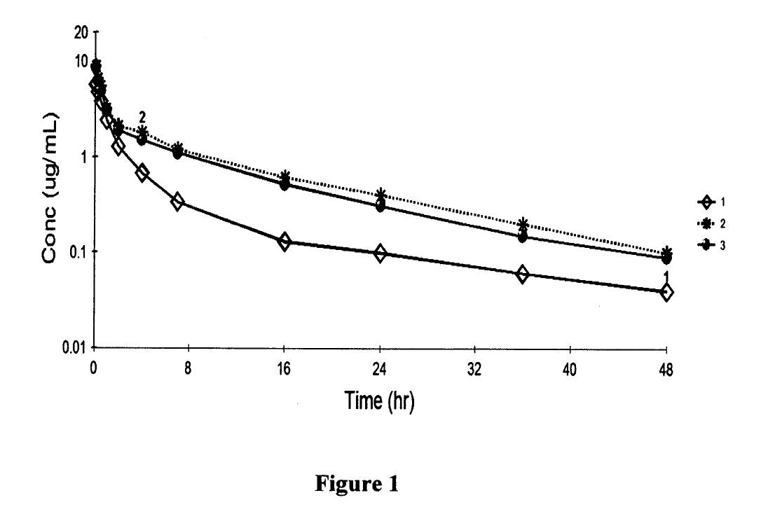

FIG. 1 shows comparison among blood concentrations of various rapamycin formulations administered intravenously. The AUC (area under the curve) of both the DAG-PEG (GDO-12) lipid formulations (2) of rapamycin-cyclosporine and (3) of rapamycin were 2 to 2.5-fold higher than the control formulation (1) of rapamycin (Table 7).

TABLE 7

| | | GDO-12 of | |
|---|---|---|---|
| | The control | rapamycin- | GDO-12 of |
| PK Parameters | formation | cyclosporine | rapamycin |
| AUC_{0-48 h} (μg h/mL) | 12.833 ± 1.501 | 31.181 ± 0.904 | 27.611 ± 0.954 |
| AUC_{inf} (μg h/mL) | 12.900 ± 0.754 | 33.196 ± 0.808 | 29.162 ± 0.990 |
| V (l/kg) | 10.384 ± 1.459 | 6.069 ± 0.296 | 5.318 ± 0.381 |
| CL(L/h kg) | 0.775 ± 0.090 | 0.301 ± 0.009 | 0.342 ± 0.011 |
| Kₑₗ (h⁻¹) | 0.075 ± 0.001 | 0.050 ± 0.002 | 0.065 ± 0.041 |
| t_{1/2} (h) | 9.285 ± 0.259 | 13.966 ± 0.744 | 10.750 ± 0.597 |
| MRT_{0-48 h} (h) | 4.326 ± 1.539 | 10.703 ± 1.727 | 10.394 ± 0.858 |

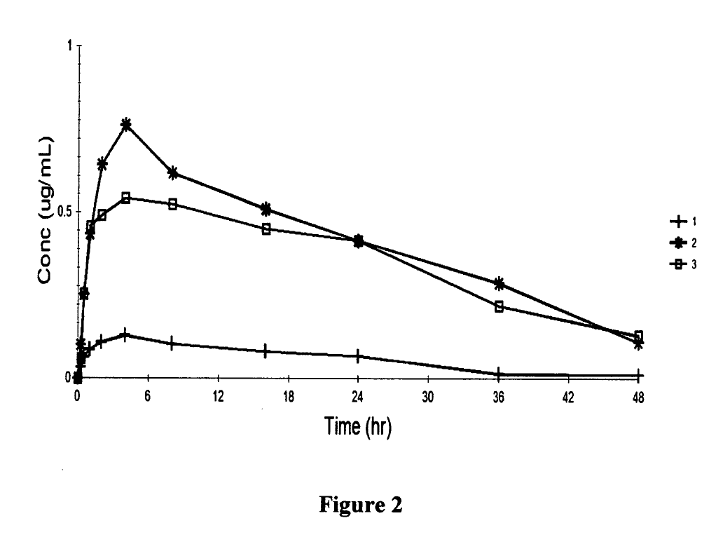

FIG. 2 shows a comparison among oral administrations of (1) the control formulation of rapamycin, DAG-PEG (GDO-12) lipid formulations (2) of rapamycin-cyclosporine and (3) of rapamycin. Bioavailability of the DAG-PEG formulations were about 63% (2) and 62% (3) versus about 27% for the control formulation (1). However the enhancement of cyclosporine on bioavailability of rapamycin was only marginal and no significant slow down on the absorption (Table 8). This may be explained that the enhancement of the rapamycin solubility by DAG-PEG (GDO-12) lipid is the dominating effect on the absorption of rapamycin as compared to the inhibition of cyclosporine on CYP3A4 and P-glycoprotein.

TABLE 8

| | | GDO-12 of | |
|---|---|---|---|
| | The control | rapamycin- | GDO-12 of |
| PK Parameters | formation | cyclosporine | rapamycin |
| AUC_{0-48 h} (μg h/mL) | 3.455 ± 0.407 | 20.341 ± 1.578 | 17.109 ± 1.604 |
| AUC_{inf} (μg h/mL) | 5.932 ± 0.457 | 22.490 ± 1.547 | 19.790 ± 1.676 |
| V (L/kg) | 77.345 ± 0.787 | 11.117 ± 0.201 | 14.549 ± 1.037 |
| CL(L/h kg) | 1.685 ± 0.112 | 0.444 ± 0.007 | 0.600 ± 0.019 |
| Kₑₗ (h⁻¹) | 0.022 ± 0.002 | 0.040 ± 0.002 | 0.048 ± 0.030 |
| t_{1/2} (h) | 31.804 ± 2.928 | 17.331 ± 2.023 | 16.804 ± 1.874 |
| Bioavailability (%)¹ | 26.920 ± 2.421 | 65.24 ± 2.164 | 61.964 ± 1.459 |
| MRT_{0-48 h} (h) | 19.730 ± 4.561 | 18.313 ± 2.988 | 18.854 ± 1.556 |

¹bioavailability is calculated based on the AUC_{0-48 h} of the corresponding formula.

While preferred embodiments of the present invention have been described, those skilled in the art will recognize that other and further changes and modifications can be made without departing from the spirit of the invention, and all such changes and modifications should be understood to fall within the scope of the invention.

We claim:

1. A method of treating a disease in a mammal, the method comprising;
preparing a composition comprising a solubilizing agent; a solidifying agent; rapamycin; and a capsule; and
administering the composition to the mammal orally.

2. The method of claim 1, where the solubilzing agent comprises a DAG-PEG.

3. The method of claim 2, where the MW of the PEG chain is less than about 600.

4. The method of claim 2, where the melting point of the DAG-PEG is less than about 25 degrees C.

5. The method of claim 1, where the solidifying agent comprises a DAG-PEG.

6. The method of claim 5, where the MW of the PEG chain is greater than about 600.

7. The method of claim 5, where the melting point of the DAG-PEG is greater than about 35 degrees C.

8. The method of claim 1, where the composition further comprises cyclosporin.

9. The method of claim 1, where DAG-PEGS comprise about 90 to 99.8 weight percent of the capsule content.

10. The method of claim 1, where the capsule contains between about 0.5 and 25 mg of rapamycin.

11. The method of claim 8, where the capsule contains between about 0.5 and 10 mg of rapamycin.

12. The method of claim 8, where the weight ratio of cyclosporin to rapamycin is between about 0.1 to 1.0.

13. The method of claim 1, where the disease is cancer.

14. The method of claim 1, where the disease is a fungal infection.

15. The method of claim 1, where treatment of the disease requires immunosuppression.
